(19) United States
(12) Patent Application Publication
(10) Pub. No.: US 2020/0368428 A1
(43) Pub. Date: Nov. 26, 2020

(54) HERMETIC SEAL FOR USE IN AN IMPLANTABLE METRONOMIC DRUG PUMP AND A MEHTOD OF MANUFACTURING THE SAME

(71) Applicant: Cognos Therapeutics Inc., Inglewood, CA (US)

(72) Inventors: Winston Wu, Alhambra, CA (US); Darren W. Branch, Albuquerque, NM (US); Jay Bischoff, Albuquerque, NM (US)

(73) Assignee: Cognos Theraeutics Inc, Inglewood, CA (US)

(21) Appl. No.: 16/985,075

(22) Filed: Aug. 4, 2020

Related U.S. Application Data
(63) Continuation-in-part of application No. 16/397,623, filed on Apr. 29, 2019.

(51) Int. Cl. A61M 5/142; A61K 31/519

(52) U.S. Cl. CPC A61M 5/14276; A61M 2207/00; A61M 2205/0294; A61K 31/519

(57) ABSTRACT

A method of hermetically bonding components of an implantable pump made of biocompatible materials comprises the steps of providing a first SiO₂ layer of predetermined first thickness onto a selected bonding surface of a first biocompatible component of the implantable pump to reduce the surface roughness, Ra, available for bonding below a first predetermined magnitude; providing a second SiO₂ layer of predetermined second thickness onto a selected bonding surface of a second biocompatible component of the implantable pump to reduce the surface roughness of the selected bonding surface; and bringing the first and second SiO₂ layers into contact with each other at a low temperature with a low pressure to form a high quality hermetic bond and seal between first and second SiO₂ layers. The invention includes an implantable pump having a hermetic seal manufactured by the method.

This application is a continuation in part and claims priority to, and the benefit of the earlier filing date of: US patent application entitled, Method and Apparatus for a Long-Term, Fully Implantable MRI Compatible Drug Pump, filed on 29 Apr. 2019, Ser. No. 16/397,623, pursuant to 35 USC 120 the contents of all of which are incorporated herein by reference and hereinafter referenced as “incorporated specifications”.

### BACKGROUND

### Field of the Technology

The present invention relates generally to implantable medical devices, and more particularly to an improved, MRI compatible and hermetically sealed, infusion pump incorporating a piezoelectric pump mechanism for use in local administration of biological response modifiers and chemotherapeutic agents in tumor fighting.

### Description of the Prior Art

The underlying hypothesis of using cytotoxic drug is that more is better. Thus, a first step in administrating a cytotoxic agent is to determine the maximum tolerated dose (MTD). However, when used in traditional treatment modes, such as chemotherapy, the cytotoxic agents are delivered to the patient in a manner that allows the cytotoxic agents to be distributed systemically throughout the body of the patient. Relatively large doses of the drugs are required since only a small fraction of the administered dose will be present at the tumor site at any given time. The remainder of the dose will be in other parts of the body. Moreover, a major problem with conventional chemotherapy is the lack of specificity of the cancer cell.

The use of large doses of toxic agents often leads to serious and debilitating side effects. Moreover, the global administration of drugs is often not compatible with combination therapies where several medicating agents are used synergistically to treat tumors or other conditions. Thus, the global or systemic administration of medicating agents to treat tumors and other such medical conditions is an inefficient, often dangerous, technique that often leads to severe or debilitating side effects.

The current generation of infusion pumps in market place has five limitations. First, they use electromagnetic pump systems whose operations are sensitive to intense magnetic radiation from magnetic resonance imaging (MRI). The use of MRI is routine for cancer patients, so these pumps are not suitable for cancer patients. Second, these infusion pumps have peristaltic pumping mechanism that is prone to leaking from the flexible tubing. This peristaltic pumping mechanism cannot be hermetically sealed, leading to corrosion inside the pumps after long implantation times Third, these pumps do not have wireless communication for monitoring and adjustment of dosage. The delivery rate is set mechanically at the time of implantation. Fourth, these pumps do not have feedback sensors to monitor the concentration of medication after delivery. The rate of delivery is constant and cannot be adjusted based on how well medication is absorbed at the point of delivery.

Finally and fifth, the implanted infusion pumps are intended to remain implanted in the body for an extended period of time, typically for the remaining life expectancy of the patient, but this goal has not always been achievable. The pump is subjected to long term exposure in a semiliquid environment of body fluids, motion, and immunological reactions. Such environments have been found to be corrosive or oxidative on implant components with the result that prior infusion pumps tend to eventually leak body fluids into the pump interior, where metallic or polymeric elements in the pumping mechanism, and/or control circuitry become corroded, oxidized, degrade or lose their elasticity. Attempts to provide long term sealed enclosures for such implanted pumps have not been totally successful or reliable, even in instances where an attempt has been made to encase the mechanism and circuitry in conventional hermetically sealed containments.

Achieving a hermetic bond using biocompatible materials is necessary in an implanted medical devices. The challenge is to make a low temperature hermetic bond between a titanium reservoir to be filled with an anticancer drug and a silicon based micropump. The problem is conventional hermetic bonding methods use non-biocompatible eutectic solders such as Ag/Sn, Au/Sn, with pre-deposited Ti/Ni/Au layers or Al/Ge to overcome the inherent surface roughness and planarity of metal parts such as titanium. Even if high temperature bonding methods are biocompatible, they lead to excessive thermal expansion and stress for dissimilar materials. For instance, the Al/Ge system requires temperatures in excess of 410° C. to complete the bond leading to possible bond failure. For Si and Ti parts bonded at 410° C., where the thermal expansion coefficients are 4 ppm/m and 9 ppm/m ° C., respectively, the final stress in the bond at room temperature is a high risk failure point.

Although eutectic materials such as Ag/Sn form hermeticbonds at a much lower temperatures around 210° C., they are not suitable for implanted medical devices.

What is needed is a new way of providing a hermetic seal which is not subject to the limitations and failure rates of conventional hermetic seals.

### BRIEF SUMMARY

The illustrated embodiments of the invention include a method of hermetically bonding components of an implantable pump made of biocompatible materials comprising the steps of: providing a first SiO₂ layer of predetermined first thickness onto a selected bonding surface of a first biocompatible component of the implantable pump to reduce the surface roughness, Ra; available for bonding below a first predetermined magnitude: providing a second SiO₂ layer of predetermined second thickness onto a selected bonding surface of a second biocompatible component of the implantable pump to reduce the surface roughness of the selected bonding surface; and bringing the first and second SiO₂ layers into contact with each other at a low temperature with a low pressure to form a high quality hermetic bond and seal between first and second SiO₂ layers.

Where the first biocompatible component is composed of silicon, and where the second biocompatible component is composed of titanium, the step of providing a first SiO₂ layer onto the bonding surface of the first biocompatible component includes the step of providing the first SiO₂ layer as a native SiO₂ layer of the order of 2 nm thickness (defined in this specification as 0.2 nm to 20 nm) onto silicon bonding surface of the first biocompatible component. The step reduces the surface roughness, Ra below approximately 0.5 nm or less (defined in this specification as 1 nm-0.1 nm).

Where the first biocompatible component is composed of silicon, and where the second biocompatible component is composed of titanium, the step of providing a second S. 02 layer of predetermined second thickness onto a selected bonding surface of a second biocompatible component of the implantable pump to reduce the surface roughness of the selected bonding surface includes the step of providing a second SiO₂ layer of the order of 1 to 1.5 μm thickness (defined in this specification as 0.15 to 15 μm) onto a selected bonding surface of the second biocompatible titanium component of the implantable pump to reduce the surface roughness.

The method further includes the step of chemical mechanical polishing the selected bonding surface of the second SiO₂ layer of the second biocompatible titanium component of the implantable pump before bringing the first and second SiO₂ layers into contact with each other.

The method further includes the step of providing the second biocompatible titanium component of the implantable pump with a native titanium oxide layer of the order of 50 nm thickness (defined in this specification as 5 to 500 nm) before providing the second SiO₂ layer of predetermined second thickness.

The step of providing a second SiO₂ layer of predetermined second thickness onto a selected bonding surface of a second biocompatible component of the implantable pump to reduce the surface roughness of the selected bonding surface includes the step of providing the second SiO₂ layer of the order of 1.5 μm thickness (defined in this specification as 0.15 to 15 μm) onto a selected bonding surface of the second biocompatible titanium component of the implantable pump to reduce the surface roughness.

The step of bringing the first and second SiO₂ layers into contact with each other at a low temperature includes the step of bringing the first and second SiO₂ layers into contact with each other at approximately 100° C. or less (defined in this specification as 150-50° C.).

The step of bringing the first and second SiO₂ layers into contact with each other with a low pressure includes the step of bringing the first and second SiO₂ layers into contact with each other with a few grams (0.1 to 10 grams) pressure or the weight of the first or second biocompatible component.

The scope of the illustrated embodiments also extends to a hermetic seal of a biocompatible implantable pump between a first and second biocompatible component. The seal includes: a first SiO₂ layer of a predetermined first thickness disposed onto a selected bonding surface of the first biocompatible component of the implantable pump to reduce the surface roughness, Ra, available for bonding below a first predetermined magnitude; a second SiO₂ layer of predetermined second thickness disposed onto a selected bonding surface of the second biocompatible component of the implantable pump to reduce the surface roughness, where the first and second SiO₂ layers are brought into contact with each other at a low temperature ((defined in this specification as 150-50° C.) with a low pressure (defined in this specification as 10-1 gm) to form a high quality hermetic bond and seal between first and second SiO₂ layers.

The implantable pump is a piezoelectric pump with a piezoelectric membrane included in the first biocompatible component and has a titanium mount plate included in the second biocompatible component. The first SiO₂ layer is of the order of 2 nm thickness (defined in this specification as 0.2 nm to 20 nm) and is disposed onto a bonding surface of the first biocompatible component of the implantable pump to reduce the surface roughness, Ra, available for bonding below approximately 0.5 nm or less (defined in this specification as 0.55 nm or less). A titanium oxide layer of the order of 50 nm thickness (defined in this specification as 5-500 nm) is disposed on the titanium mount plate. The second SiO₂ layer is of the order of 1.5 μm thickness (defined in this specification as 0.15-15 μm) and is disposed onto the titanium oxide layer of the second biocompatible component of the implantable pump. The second. SiO₂ layer is chemical mechanical polished, and the first and second SiO₂ layers are brought into contact with each other at approximately 100° C. or less (defined as 150-50° C.) with a pressure of a few grams (defined in this specification as 1-10 gm) or the weight of the first or second biocompatible components to form a high quality hermetic bond and seal between first and second SiO₂ layers.

The first biocompatible component is composed of silicon, the second biocompatible component is composed of titanium, and the first SiO₂ layer has a predetermined first thickness and is disposed onto a selected bonding surface of a first biocompatible component of the implantable pump to reduce the surface roughness, Ra, available for bonding below a first predetermined magnitude, and where the first SiO₂ layer is a native SiO₂ layer of the order of 2 nm thickness (defined in this specification as 0.2-20 nm) and is disposed onto silicon bonding surface of the first biocompatible component of the implantable pump to reduce the surface roughness, Ra, available for bonding below approximately 0.5 nm or less (defined in this specification as 0.55 nm or less).

Stated alternatively, the first biocompatible component s composed of silicon, the second biocompatible component is composed of titanium; and the second SiO₂ layer has a predetermined second thickness and is disposed onto a selected bonding surface of a second biocompatible component of the implantable pump to reduce the surface roughness of the selected bonding surface, where the second SiO₂ layer is of the order of 1.5 μm thickness (defined in this specification as 0.15 to 15 μm) and is disposed onto a selected bonding surface of the second biocompatible titanium component of the implantable pump to reduce the surface roughness.

The selected bonding surface of the second SiO₂ layer of the second biocompatible titanium component of the implantable pump is chemical mechanical polished before the bringing the first and second SiO₂ layers into contact with each other.

The second biocompatible titanium component of the implantable pump includes a native titanium oxide layer of the order of 50 nm thickness (defined in this specification as 5-500 nm) for disposition with the second SiO₂ layer of predetermined second thickness.

The second SiO₂ layer of predetermined second thickness is disposed onto a selected bonding surface of a second biocompatible component of the implantable pump, and is of the order of 1.5 μm thickness (defined in this specification as 0.15 to 15 μm) and is disposed onto a selected bonding surface of the second biocompatible titanium component of the implantable pump to reduce the surface roughness.

The first and second SiO₂ layers re brought into contact with each other at a low temperature (defined in this specification as 150-50° C.) with a low pressure with a few grams force (defined in this specification as 1-10 gm) or the weight of the first or second biocompatible component.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112, The disclosure can be better visualized by turning now to the following drawings wherein like elements are referenced by like numerals.

### BRIEF DESCRIPTION OF THE DRAWINGS

FIG. 1 is a top perspective view of the piezoelectric pump 10 of the illustrated embodiment of the invention.

FIG. 2 is an exploded perspective view of pump of FIG. 1.

FIG. 3 is a cross-sectional perspective view of the assembled pump of FIGS. 1 and 2,

FIG. 4 is a cross-sectional perspective view of the assembled similar to FIG. 3 except that the piezoelectric chip, piezoelectric membrane, and hermetic mount plate are shown as combined into a subassembly apart from fluid plate.

FIG. 5 is a top perspective view of the chip, piezoelectric membrane, and hermetic mount plate combined.

FIG. 6 is a bottom perspective view of hermetic mount plate and illustrates a defined outlet cavity in the plate and a defined inlet cavity in the plate.

FIG. 7 is a diagrammatic illustration of the method by which the chip is hermetically sealed or bonded to hermetic mount plate.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

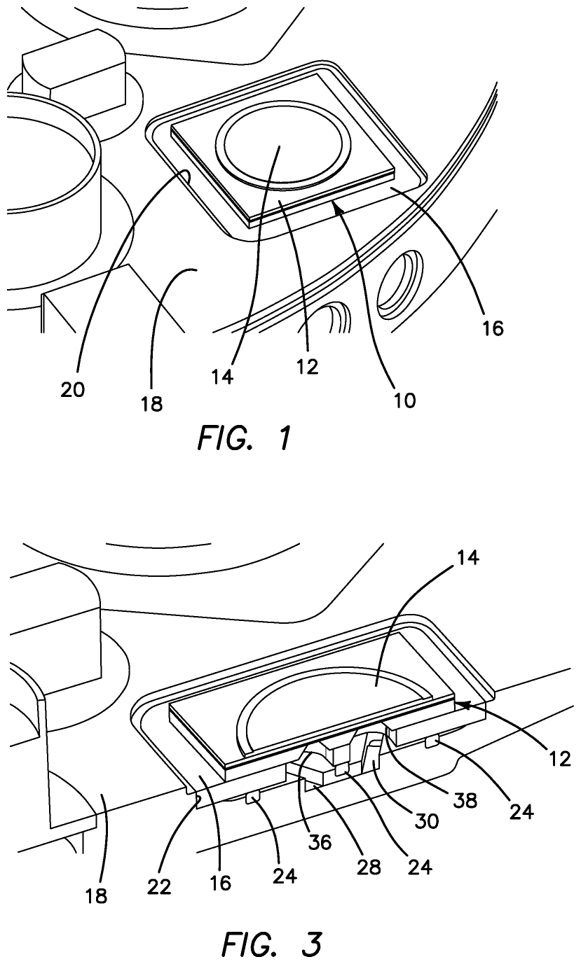

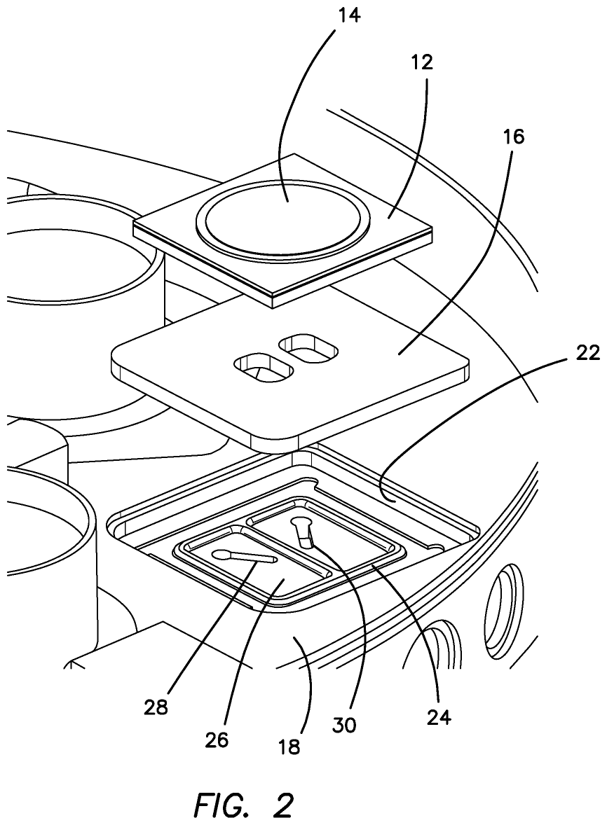

FIG. 1 is a top perspective view of the piezoelectric pump 10 of the illustrated embodiment of the invention. Pump 10 is mounted on an 8 mm×8 mm Si chip 12 on which a circular piezoelectric membrane 14 is mounted. Chip 12 is hermetically sealed and bonded to a titanium hermetic mount plate 16 as described in greater detail below. Pump 10 is disposed on a metal fluid plate 18 of a middle layer of the implanted pump system as shown and described in FIG. 2 of the incorporated specifications. A 10 mm×10 mm×0.64 mm titanium hermetic mount plate 16 is, when assembled, laser welded to metal fluid plate 18 along its entire joint periphery 20. The system circuitry is included in an upper layer of the implanted pump system and the entire system is contained within a sealed flat cylindrical stainless steel case.

FIG. 2 is an exploded perspective view of pump 10 as seen in FIG. 1. Hermetic mount plate 16 is disposed into a mating cavity 22 defined in fluid plate 18 on top of a polymeric or rubber double ring pump seal 24 that provides for a leak tight seal between the bottom of plate 16 and floor 26 of cavity 22. An outlet orifice 28 is defined in one side of floor 26 encircled by one half of double ring pump seal 24 and an inlet orifice 30 is defined in the other side of floor 26 encircled by the other half of double ring pump seal 24.

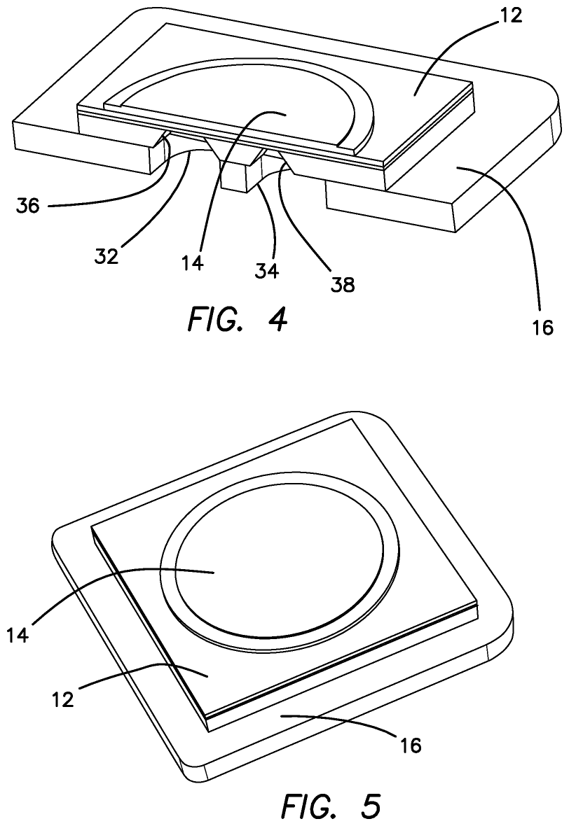

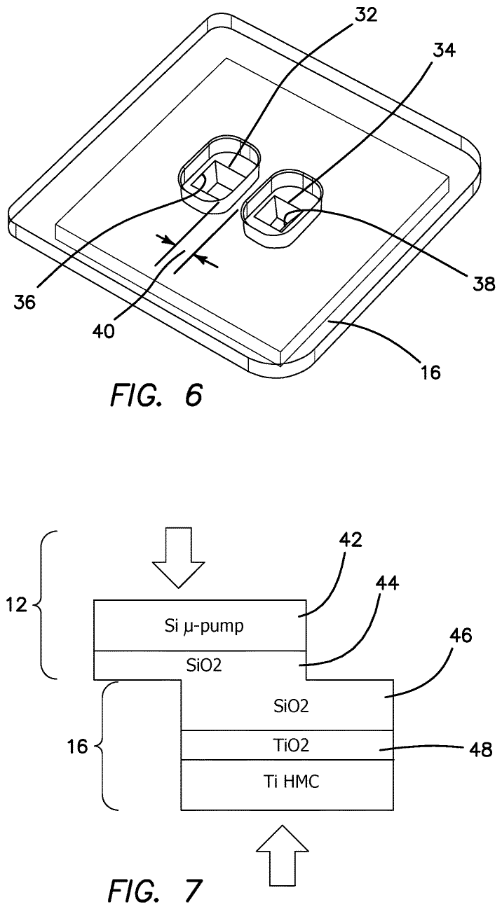

FIG. 3 is a cross-sectional perspective view of the assembled pump 10 of FIGS. 1 and 2, which illustrates how the various features of chip 12, piezoelectric membrane 14, hermetic mount plate 16, fluid plate 18, cavity 22, pump seal 24, outlet 28 and inlet 30 align and combine to form the completed pump 10. FIG. 4 is a cross-sectional perspective view of the assembled pump 10 similar to FIG. 3 except that chip 12, piezoelectric membrane 14, and hermetic mount plate 16 are shown as combined into a subassembly apart from fluid plate 18. The perspective view of FIG. 5 illustrates a top perspective view of chip 12, piezoelectric membrane 14, and hermetic mount plate 16 combined. FIG. 6 is a bottom perspective view of hermetic mount plate 16 and illustrates a defined outlet cavity 32 in plate 16 and a defined inlet cavity 34 in plate 16. Also shown in FIG. 6 is a pump outlet chamber 36 defined into overlying layer 12 aligned with outlet cavity 32 and a pump inlet chamber 38 defined into overlying layer 12 aligned with outlet cavity 34. Outlet cavity 32 and inlet cavity 34 are defined in plate 16 and separated from each other by an inlet/outlet separation distance 40. The alignment and structural relationship of pump outlet chamber 36 defined into overlying layer 12, outlet cavity 32, pump inlet chamber 38 defined into overlying layer 12 and outlet cavity 34 can be better seen in the cross-sectional views of FIGS. 3 and 4.

The pump structure now having been generally described, turn and consider the hermetic bonding of Ti hermetic plate 16 to the underside of chip 12. The Ti surface of plate 16 is too rough for a direct bond to Si and SiO₂. Instead we added 1.5 um of silicon dioxide layer 46 onto the Ti of plate 16 to reduce the surface roughness below 1 nm to achieve a low temperature hermetic bond as shown in FIG. 7. To develop a biocompatible hermetic bond at low temperature, we relied on titanium, a common biomedical material used in implants and silicon dioxide for the bonding process. The drug reservoir was created from titanium to hold the drug methotrexate (i.e. commonly used to treat cancer of the breast, skin, head and neck, or lung). A hermetic bond will easily form when both mating surfaces have a surface roughness less than 1 nm. Attempts to use chemical mechanical polishing (CMP) of titanium produced a surface roughness ˜10-20 nm, which was too rough for a hermetic bond to form. This problem was overcome by depositing a thin layer 46 of silicon dioxide on to the titanium reservoirs mating surface of plate 16 then use CMP to form a new low roughness surface for hermetic bonding to the silicon dioxide micropump surface 44.

The conventional CMP process uses an abrasive and corrosive chemical slurry (commonly a colloid) in conjunction with a polishing pad and retaining ring, typically of a greater diameter than the wafer or substrate. The pad and wafer are pressed together by a dynamic polishing head and held in place by a plastic retaining ring. The dynamic polishing head is rotated with different axes of rotation (i.e., not concentric). This removes material and tends to even out any irregular topography, making the wafer flat or planar. This may be necessary to set up the wafer for the formation of additional circuit elements. For example, CMP can bring the entire surface within the depth of field of a photolithography system, or selectively remove material based on its position. Typical depth-of-field requirements are down to angstrom levels for the latest 22 nm technology.

The method of forming the hermetic sealing between chip 12 carrying the piezoelectric membrane 14 and hermetic mount plate 16 is unconventional in order to achieve long term sealing and operability of the pump 10. FIG. 7 is a diagrammatic illustration of the method by which chip 12 is hermetically sealed or bonded to hermetic mount plate 16. FIG. 7 shows only the portion of underlying Si pump layer 42 of chip 12 into which pump outlet chamber 36 and pump inlet chamber 38 are defined (not shown in FIG. 7). It is to be understood that the remaining layers and structures of chip 12, including piezoelectric membrane 14, are carried above layer 42 shown in FIG. 7. The bottom surface of layer 42 is provided with a 2 nm thick native silicon oxide layer 44 with a prepared roughness average (Ra), defined in this specification as an average distance or height of the peaks and valleys from a mean line, of less than 0.5 nm. Similarly, the opposing upper surface of hermetic mount plate 16 is a prepared 1.5 μm thick silicon oxide layer 46 that is chemical mechanical polished. Below layer 46 is a 50 nm thick native titanium oxide layer 48 on the top surface of titanium hermetic mount plate 16. The means of disposing the titanium oxide layer 48 onto layer 64 for planarization may be accomplished a conventional spin-on-glass technique (See Interconnect. Dielectrics, Application Note No. 1, Honeywell) as well as chemical vapor deposition. When the surfaces of SiO₂ layers 44 and 46 are brought into contact at approximately 100° C. with a low pressure, e.g. a few grams or just the weight of the components themselves, a high quality hermetic bond and seal is formed between layers 44 and 46.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim, Although elements may be described above as acting in certain combinations and even initially claimed as such, it is to be expressly understood that one or more elements from a claimed combination can in some cases be excised from the combination and that the claimed combination may be directed to a subcombination or variation of subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A method of hermetically bonding components of an implantable pump made of biocompatible materials comprising:
providing a first SiO₂ layer of predetermined first thickness onto a selected bonding surface of a first biocompatible component of the implantable pump to reduce the surface roughness, Ra, available for bonding below a first predetermined magnitude;
providing a second SiO₂ layer of predetermined second thickness onto a selected bonding surface of a second biocompatible component of the implantable pump to reduce the surface roughness of the selected bonding surface; and
bringing the first and second SiO₂ layers into contact with each other at a low temperature with a low pressure to form a high quality hermetic bond between first and second SiO₂ layers.

2. The method of claim 1 where the first biocompatible component is composed of silicon, where the second biocompatible component is composed of titanium, and where providing a first SiO₂ layer of predetermined first thickness onto a selected bonding surface of a first biocompatible component of the implantable pump to reduce the surface roughness, Ra, available for bonding below a first predetermined magnitude comprises providing the first SiO₂ layer as a native SiO₂ layer of the order of 2 nm thickness onto silicon bonding surface of the first biocompatible component of the implantable pump to reduce the surface roughness, Ra, available for bonding below approximately 0.5 nm or less.

3. The method of claim 1 where the first biocompatible component is composed of silicon, where the second biocompatible component is composed of titanium, and where providing a second SiO₂ layer of predetermined second thickness onto a selected bonding surface of a second biocompatible component of the implantable pump to reduce the surface roughness of the selected bonding surface comprises providing the second SiO₂ layer of the order of 1.5 μm thickness onto a selected bonding surface of the second biocompatible titanium component of the implantable pump to reduce the surface roughness.

4. The method of claim 3 further comprising chemical mechanical polishing the selected bonding surface of the second SiO₂ layer of the second biocompatible titanium component of the implantable pump before bringing the first and second SiO₂ layers into contact with each other.

5. The method of claim 3 further comprising providing the second biocompatible titanium component of the implantable pump with a native titanium oxide layer of the order of 50 nm thickness before providing the second SiO₂ layer of predetermined second thickness.

6. The method of claim 2 where providing a second SiO₂ layer of predetermined second thickness onto a selected bonding surface of a second biocompatible component of the implantable pump to reduce the surface roughness of the selected bonding surface comprises providing the second SiO₂ layer of the order of 1.5 μm thickness onto a selected bonding surface of the second biocompatible titanium component of the implantable pump to reduce the surface roughness.

7. The method of claim 6 further comprising chemical mechanical polishing the selected bonding surface of the second SiO₂ layer of the second biocompatible titanium component of the implantable pump before bringing the first and second SiO₂ layers into contact with each other.

8. The method of claim 6 further comprising providing the second biocompatible titanium component of the implantable pump with a native titanium oxide layer of the order of 50 nm thickness before providing the second SiO₂ layer of predetermined second thickness.

9. The method of claim 8 further comprising chemical mechanical polishing the selected bonding surface of the second SiO₂ layer of the second biocompatible titanium component of the implantable pump before bringing the first and second SiO₂ layers into contact with each other.

10. The method of claim 1 where bringing the first and second SiO₂ layers into contact with each other at a low temperature comprises bringing the first and second SiO₂ layers into contact with each other at approximately 100° C. or less.

11. The method of claim 1 bringing the first and second SiO₂ layers into contact with each other with a low pressure comprises bringing the first and second SiO₂ layers into contact with each other with a few grams pressure or the weight of the first or second biocompatible component.

12. A method where an implantable pump is a piezoelectric pump with a piezoelectric membrane included in a first biocompatible component and a titanium mount plate included in a second biocompatible component comprising:
providing a first SiO₂ layer of the order of 2 nm thickness onto a bottom bonding surface of the first biocompatible component of the implantable pump to reduce the surface roughness, Ra, available for bonding below approximately 0.5 nm or less;
providing a titanium oxide layer of the order of 50 nm thickness on the titanium mount plate;
providing a second SiO₂ layer of the order of 1.5 μm thickness onto the titanium oxide layer of the second biocompatible component of the implantable pump;
chemical mechanical polishing the second SiO₂ layer; and
bringing the first and second SiO₂ layers into contact with each other at approximately 100° C. or less with a pressure of a few grams or the weight of the first or second biocompatible components to form a high quality hermetic bond between first and second SiO₂ layers.

13. A hermetic bond of a biocompatible implantable pump between a first and second biocompatible component comprising:
a first SiO₂ layer of a predetermined first thickness disposed onto a selected bonding surface of the first biocompatible component of the implantable pump to reduce the surface roughness, Ra, available for bonding below a first predetermined magnitude;
a second SiO₂ layer of predetermined second thickness disposed onto a selected bonding surface of the second biocompatible component of the implantable pump to reduce the surface roughness; and
where the first and second SiO₂ layers are brought into contact with each other at a low temperature with a low pressure to form a high quality hermetic bond between first and second SiO₂ layers.

14. The hermetic bond of the biocompatible implantable pump of claim 13 where the implantable pump is a piezoelectric pump with a piezoelectric membrane included in the first biocompatible component and a titanium mount plate included in the second biocompatible component,
where the first SiO₂ layer is of the order of 2 nm thickness and is disposed onto a bottom bonding surface of the first biocompatible component of the implantable pump to reduce the surface roughness, Ra, available for bonding below approximately 0.5 nm or less;
where a titanium oxide layer of the order of 50 nm thickness is disposed on the titanium mount plate;
where the second SiO₂ layer is of the order of 1.5 μm thickness and is disposed onto the titanium oxide layer of the second biocompatible component of the implantable pump;
where the second SiO₂ layer is chemical mechanical polished; and
where the first and second SiO₂ layers are brought into contact with each other at approximately 100° C. or less with a pressure of a few grams or the weight of the first or second biocompatible components to form a high quality hermetic bond between first and second SiO₂ layers.

15. The hermetic bond of the biocompatible implantable pump of claim 13 where the first biocompatible component is composed of silicon, where the second biocompatible component is composed of titanium, and where the first SiO₂ layer has a predetermined first thickness and is disposed onto a selected bonding surface of a first biocompatible component of the implantable pump to reduce the surface roughness, Ra, available for bonding below a first predetermined magnitude, and where the first SiO₂ layer is a native SiO₂ layer of the order of 2 nm thickness and is disposed onto silicon bonding surface of the first biocompatible component of the implantable pump to reduce the surface roughness, Ra, available for bonding below approximately 0.5 nm or less.

16. The hermetic bond of the biocompatible implantable pump of claim 13 where the first biocompatible component is composed of silicon, where the second biocompatible component is composed of titanium, and the second SiO₂ layer has a predetermined second thickness and is disposed onto a selected bonding surface of a second biocompatible component of the implantable pump to reduce the surface roughness of the selected bonding surface, where the second SiO₂ layer is of the order of 1.5 μm thickness and is disposed onto a selected bonding surface of the second biocompatible titanium component of the implantable pump to reduce the surface roughness.

17. The hermetic bond of the biocompatible implantable pump of claim 16 where the selected bonding surface of the second SiO₂ layer of the second biocompatible titanium component of the implantable pump is chemical mechanical polished before the bringing the first and second SiO₂ layers into contact with each other.

18. The hermetic bond of the biocompatible implantable pump of claim 16 where the second biocompatible titanium component of the implantable pump includes a native titanium oxide layer of the order of 50 nm thickness for disposition with the second SiO₂ layer of predetermined second thickness.

19. The hermetic bond of the biocompatible implantable pump of claim 15 where the second SiO₂ layer of predetermined second thickness is disposed onto a selected bonding surface of a second biocompatible component of the implantable pump, and is of the order of 1.5 μm thickness and is disposed onto a selected bonding surface of the second biocompatible titanium component of the implantable pump to reduce the surface roughness.

20. The biocompatible implantable pump of claim 13 where the first and second SiO₂ layers are brought into contact with each other at a low temperature with a low pressure with a few grams pressure or the weight of the first or second biocompatible component.
